(19) United States
(12) Patent Application Publication
(10) Pub. No.: US 2021/0236330 A1
(43) Pub. Date: Aug. 5, 2021

(54) Health Device

(71) Applicant: Cheng-Chuan Yang, Taichung City (TW)

(72) Inventor: Cheng-Chuan Yang, Taichung City (TW)

(21) Appl. No.: 16/782,062

(22) Filed: Feb. 5, 2020

(51) Int. Cl. A61F 7/02; A61F 7/00

(52) U.S. Cl. CPC A61F 7/02; A61F 2007/0075; A61F 2007/0219; A61F 7/007

(57) ABSTRACT

A health device provides cold compression and hot compression and comprises a device body, at least one temperature adjusting device, and a control module. The at least one temperature adjusting device comprises a thermoelectric cooling chip, a thermal conduction body and a heatsink. The thermoelectric cooling chip is electrically connected to the control module, and configured to convert electrical power into heating energy or cooling energy, and conduct the heating energy or cooling energy to the heatsink through the thermal conduction body, and the heatsink is in contact with the heat dissipation body to achieve effect of adjusting temperature.

### BACKGROUND OF THE INVENTION

### 1. Field of the Invention

The present invention relates to a health device, and more particularly to a health device capable of providing effect of cold compression and hot compression.

### 2. Description of the Related Art

Cold and hot compressions are commonly used treatments nowadays, and cold and hot therapies must be used appropriately in order to have a good effect. The cold compression is a method to lower a temperature of tissue to achieve a therapeutic effect, so as to reduce amount of blood in the tissue and slow down metabolism of the tissue, thereby reducing bleeding and inflammation. Furthermore, cold compression also has a certain effect on spasms and can reduce pain. Hot compression can make local micro blood vessels dilate to accelerate blood circulation, so as to reduce inflammation, reduce swelling, reduce pain and eliminate fatigue.

When acute sport injury occurs, muscles or ligaments are severely impacted to occur tears and contusions, and microvascular hemorrhage and inflammation may also occur, and in this case, cold compression is needed to suppress bleeding and inflammation. After the swelling is slowed or stopped, hot compression can be performed to promote blood circulation and tissue metabolism, so that inflammatory substances in the affected area can be removed as soon as possible.

Commercially available dual-use cold-and-hot compression pack needs to be pre-chilled in a freezer of a refrigerator or pre-heated in a microwave oven before being used. Therefore, in order to increase in-time convenience for users, a health device of the present invention is developed.

### SUMMARY OF THE INVENTION

The present invention relates to a health device capable of providing cold compression and hot compression functions, and the health device of the present invention does not need to be pre-cooled or heated by a freezer or a microwave oven, so as to facilitate to provide in-time help in a field where no freezer and microwave oven can be provided.

The present invention provides a health device including at least one device body and a control module. The at least one device body includes a heat dissipation space enclosed in the inside thereof, a heat dissipation body disposed in the heat dissipation space, and at least one temperature adjusting device disposed thereon. A part of the at least one temperature adjusting device faces the heat dissipation space, the at least one temperature adjusting device includes a thermoelectric cooling chip, a thermal conduction body and a heatsink, the thermal conduction body is disposed between the thermoelectric cooling chip and the heatsink, the heatsink comprises a plate portion and a sink portion, a side of the plate portion is abutted with the thermal conduction body, and the sink portion is extended from other side of the plate portion and in contact with the heat dissipation body of the heat dissipation space. The control module is electrically connected to the thermoelectric cooling chip of the at least one temperature adjusting device.

In an embodiment, the thermoelectric cooling chip is electrically connected to the control module which can transfer electrical power to the thermoelectric cooling chip, and the thermoelectric cooling chip converts electrical power into heating energy or cooling energy, and transfers the heating energy or cooling energy to the heatsink, and the sink portion of the heatsink transfers the heating energy or cooling energy to the heat dissipation body of the heat dissipation space, so as to achieve the effect of adjusting temperature.

In an embodiment, the at least one device body comprises a vibrating motor disposed thereon and electrically connected to the control module. The vibrating motor of the present invention can provide effect of vibration massage.

In an embodiment, the at least one device body has a side serving as a usage surface, and other side serving as a heat dissipation surface, and the health device further comprises an electrotherapy device disposed on the usage surface of the device body and electrically connected to the control module.

### BRIEF DESCRIPTION OF THE DRAWINGS

The structure, operating principle and effects of the present invention will be described in detail by way of various embodiments which are illustrated in the accompanying drawings.

FIG. 1 is a block diagram of a health device of the present invention, wherein a thermoelectric cooling chip is electrically connected to a control module.

FIG. 2 is a perspective view of a device body of FIG. 1, wherein a heat dissipation body of the device body is liquid.

FIG. 3 is a perspective view of other surface of the device body of FIG. 2.

FIG. 4 is a schematic cross-sectional view taken along IV-IV of FIG. 2.

FIG. 5 is a schematic view of a device body wound on a user's leg for cold compression and hot compression, according to the present invention.

FIG. 6 is a block diagram of a plurality of device bodies connected to each other, according to the present invention.

FIG. 7 is a schematic view of a plurality of device bodies connected to each other on a user's back for cold compression and hot compression, according to the present invention.

FIG. 8 is a block diagram of a health device of the present invention, wherein a thermoelectric cooling chip, a heat dissipation body and a control module of the health device are electrically connected to each other.

FIG. 9 is a perspective view of a device body of FIG. 8, wherein a heat dissipation body is a fan.

FIG. 10 is a schematic cross-sectional view taken along X-X of FIG. 9.

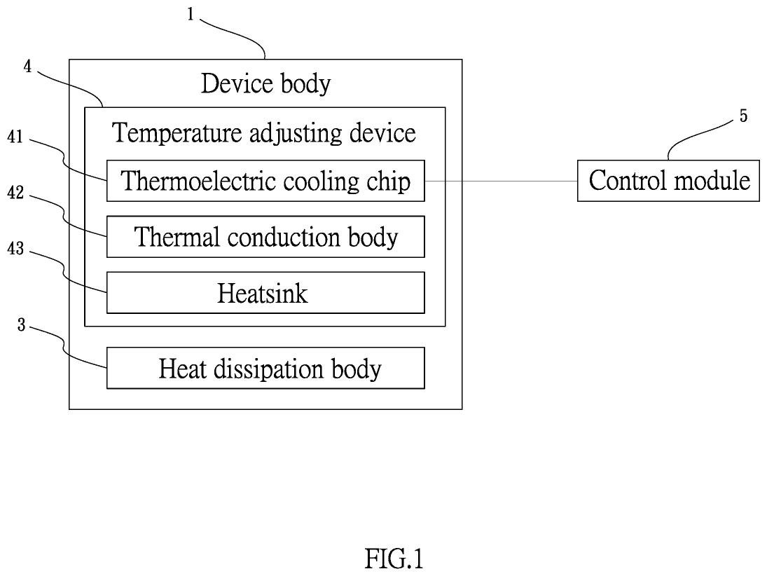

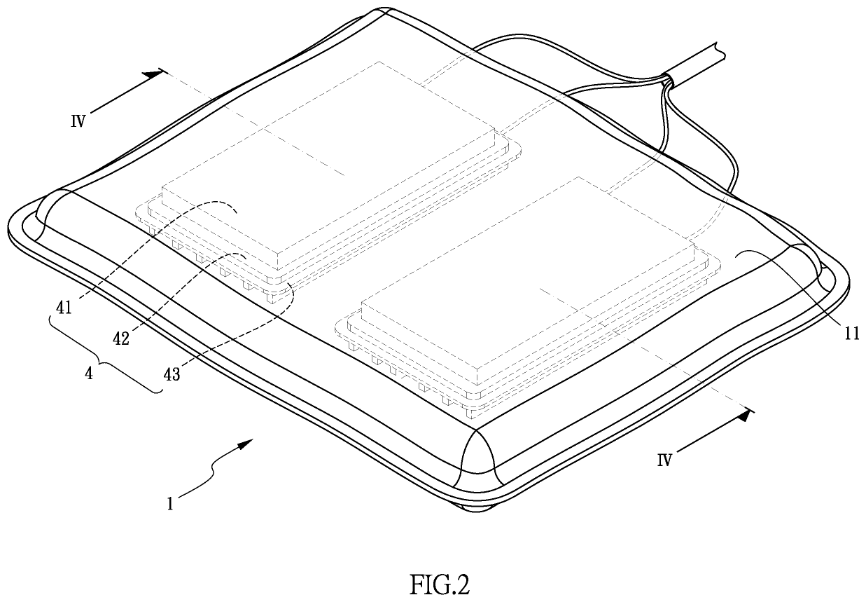

FIG. 1 is a block diagram of a health device including a device body combined with a vibrating motor electrically connected to a control module, according to the present invention.

FIG. 12 is a perspective view of the health device of FIG. 11.

FIG. 13 is a block diagram of a health device including a device body combined with an electrotherapy device, according to the present invention.

FIG. 14 is a perspective view of a health device including a device body combined with an electrotherapy device, according to the present invention.

### DETAILED DESCRIPTION OF THE PREFERRED EMBODIMENTS

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims. These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the word “comprise”, “include” and “have”, and variations such as “comprises”, “comprising”, “includes”, “including”, “has” and “having” will be acknowledged to imply the inclusion of stated elements but not the exclusion of any other elements.

The present invention relates to a health device. Please refer to FIGS. 1 to 4. The health device includes at least one device body 1, a heat dissipation space 2 enclosed in the inside of the at least one device body 1, a heat dissipation body 3 disposed in the heat dissipation space 2, and a control module 5. Each of the at least one device body 1 comprises at least one temperature adjusting device 4 disposed thereon, and a part of the at least one temperature adjusting device 4 faces towards the heat dissipation space 2. Each of the at least one temperature adjusting device 4 comprises a thermoelectric cooling chip 41, a thermal conduction body 42, and a heatsink 43. The thermal conduction body 42 is disposed between the thermoelectric cooling chip 41 and the heatsink 43, and the heatsink 43 comprises a plate portion 431 and a sink portion 432, and a side of the plate portion 431 is abutted with the thermal conduction body 42, and the sink portion 432 is extended from other side of the plate portion 431 and in contact with the heat dissipation body 3 of the heat dissipation space 2. The control module 5 is electrically connected to the thermoelectric cooling chip 41 of the at least one temperature adjusting device 4.

Each of the at least one device body 1 has at least one accommodating groove 6, and the at least one temperature adjusting device 4 is disposed in the at least one accommodating groove 6.

In an embodiment, the at least one device body 1 can be implemented by one device body 1, or by multiple device bodies 1 connected with each other. Please refer to FIGS. 1 and 5. FIG. 5 shows a schematic view of the device body 1 wound on a user's leg 9 for cold compression and hot compression, and only one device body 1 is used in the health device.

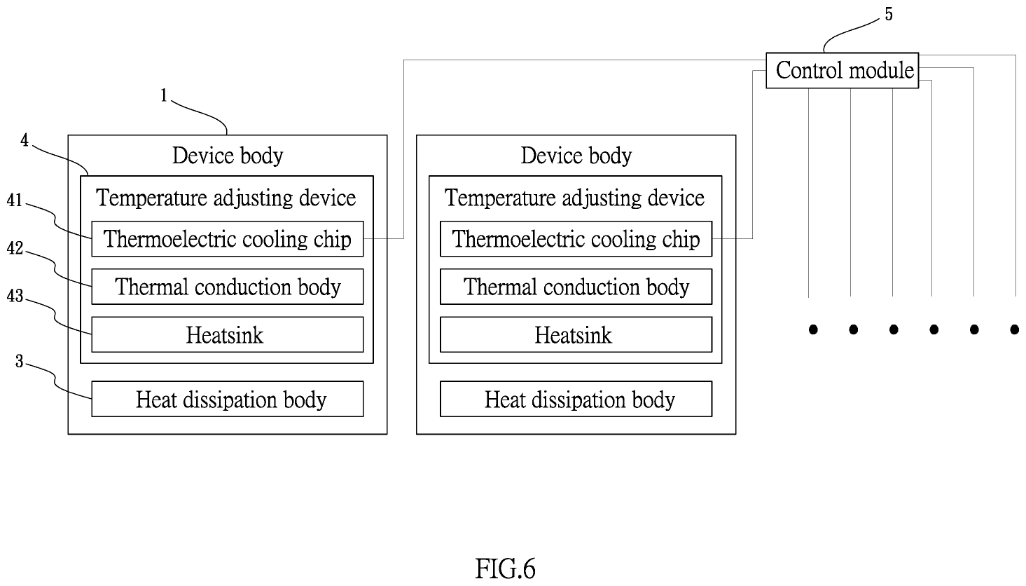

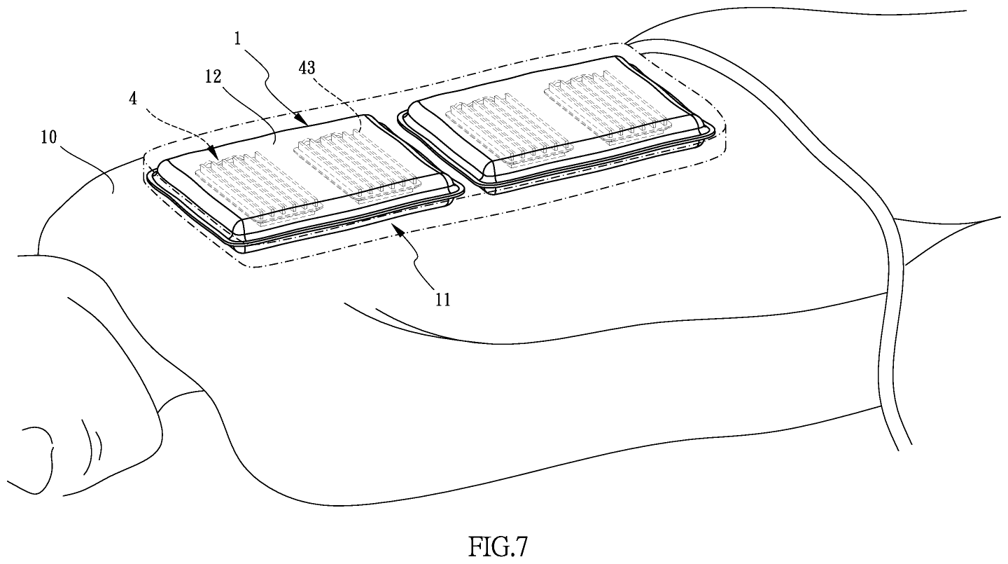

When the multiple device bodies 1 connected with each other are used in the heath device of the present invention, the multiple device bodies 1 are electrically connected to the control module 5. Please refer to FIGS. 6 and 7. In this embodiment of the present invention, two device bodies 1 are taken as an example for illustration, and FIG. 7 shows a schematic view of the two device bodies 1 placed on a back 10 of a human body for cold compression and hot compression.

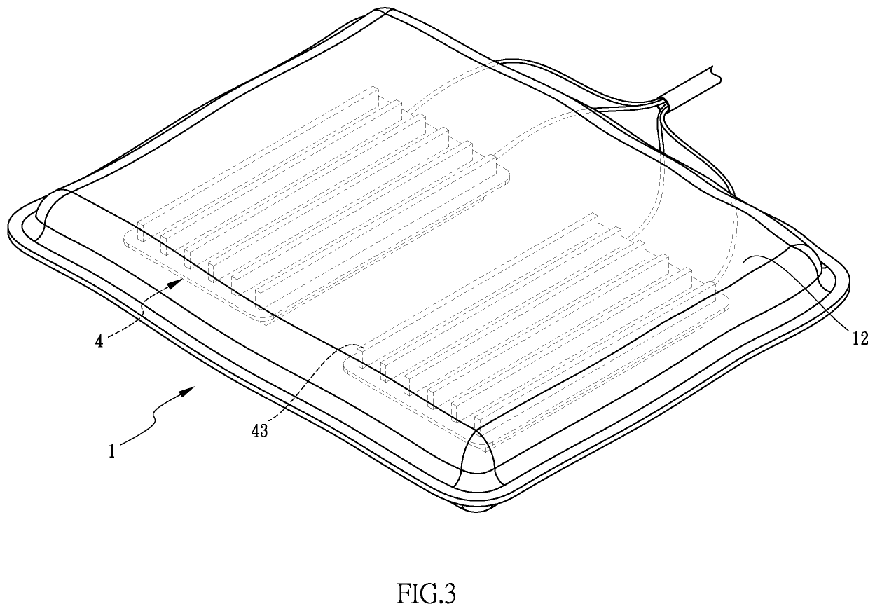

The number of the at least one temperature adjusting device 4 of the at least one device body 1 can be one or more than one, for example, FIG. 1 shows the embodiment that one temperature adjusting device 4 is used in the device body 1, and FIG. 2 shows the embodiment that more than one temperature adjusting devices 4 connected with each other are used, and two device bodies 1 are taken as an example for illustration.

In an embodiment, the thermoelectric cooling chip 41 can be a TEC chip which is a semiconductor temperature-difference thermoelectric cooling chip.

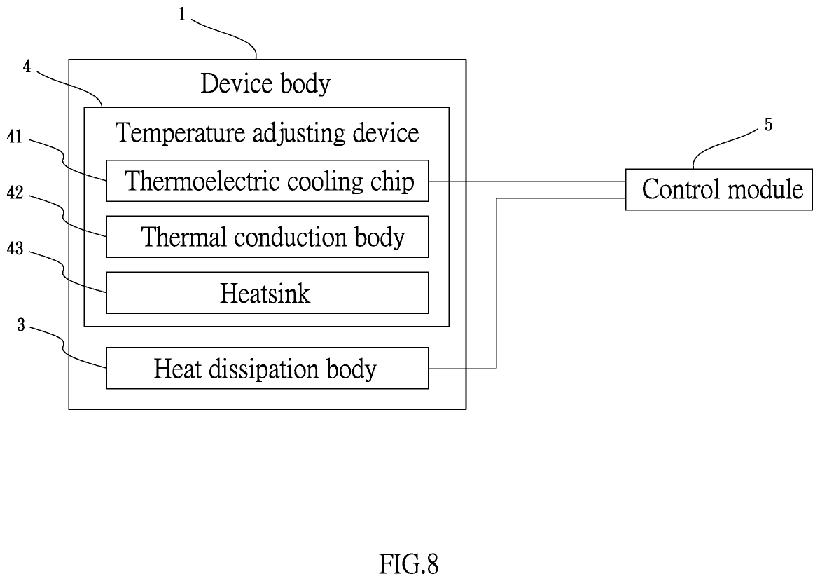

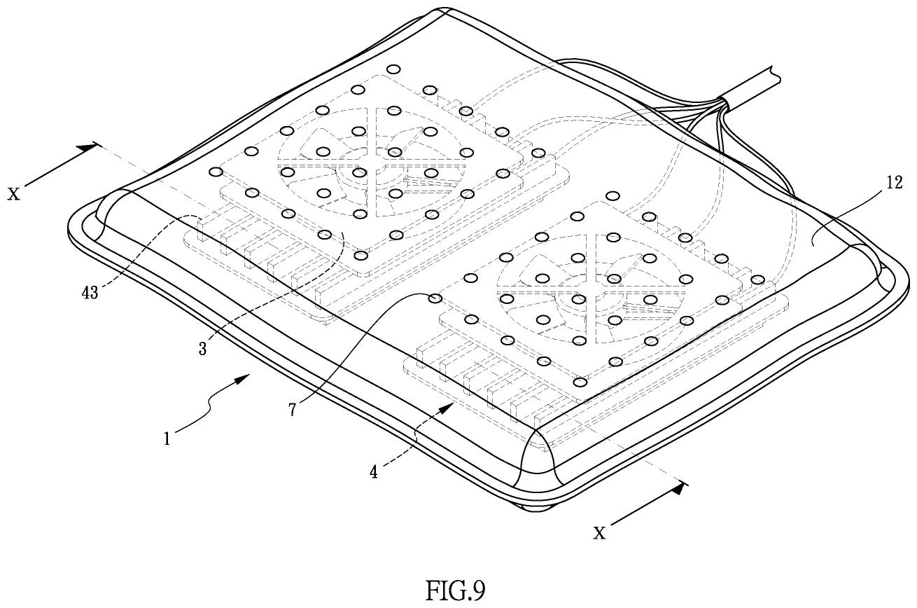

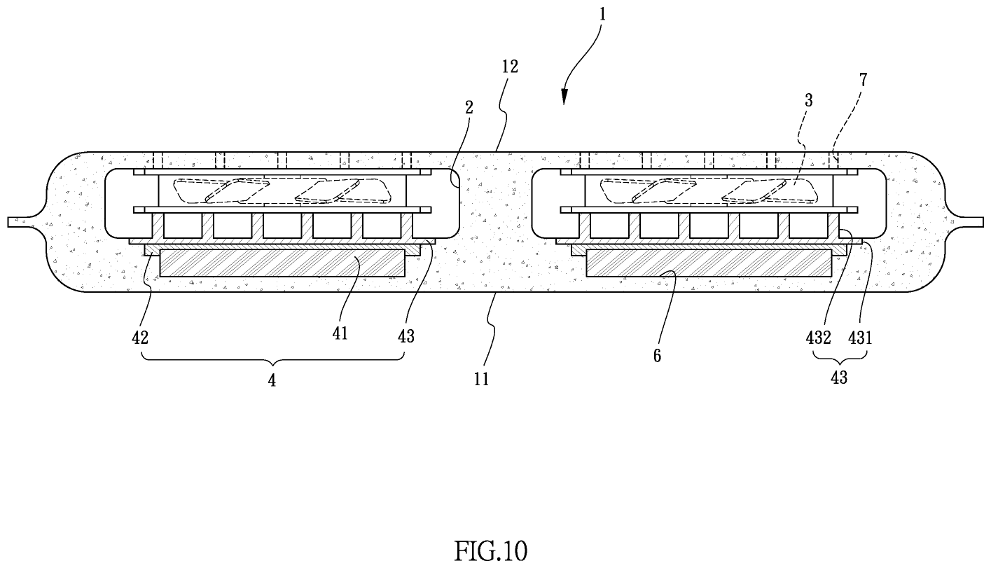

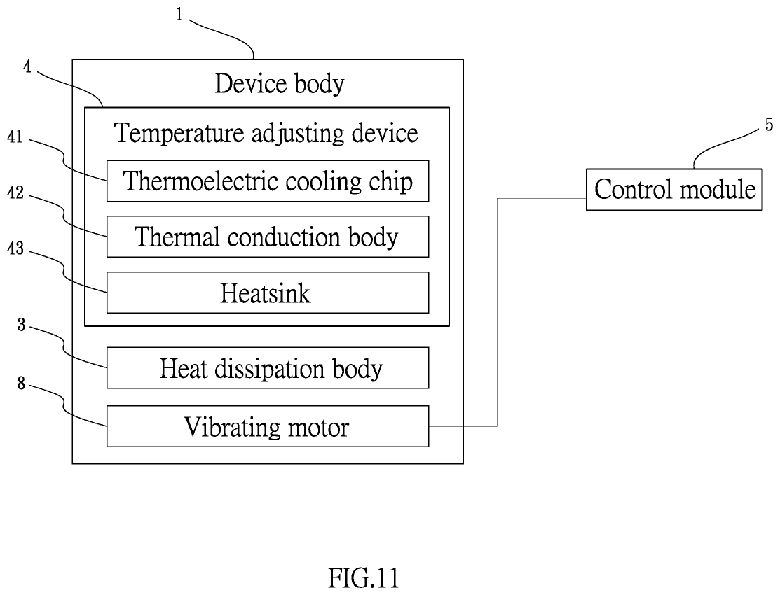

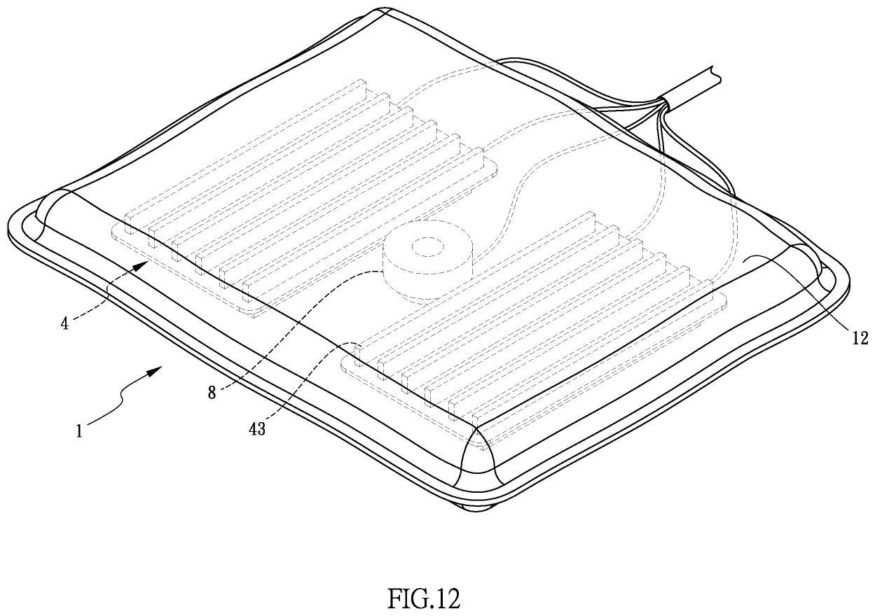

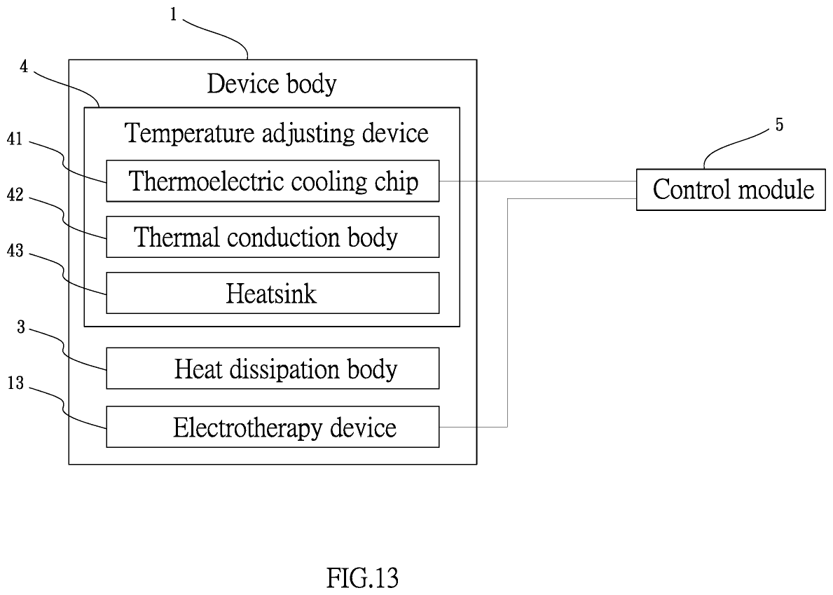

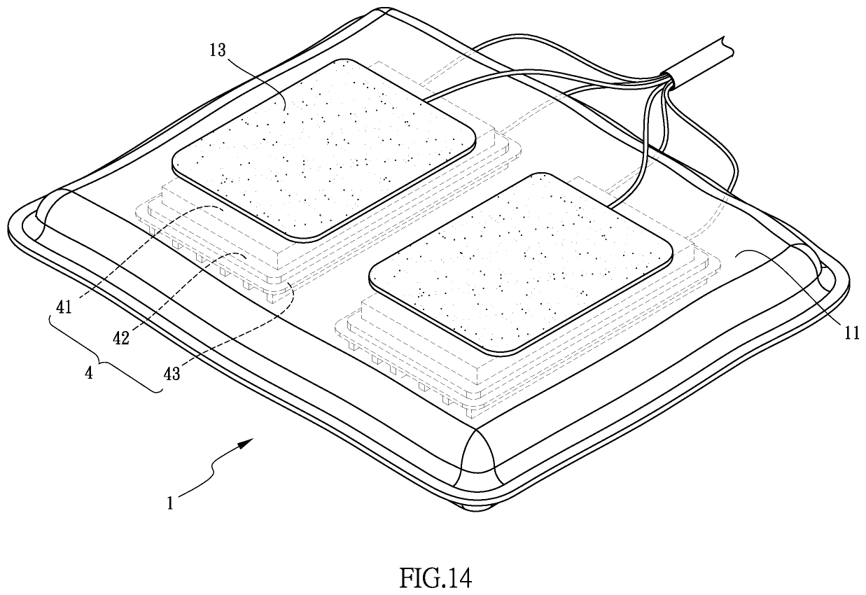

In an embodiment, the heat dissipation body 3 can be liquid, such as water, as shown in FIGS. 1 to 4. In an embodiment, the heat dissipation body 3 can be a fan, and when the heat dissipation body 3 is implemented by a fan, the at least one device body 1 comprises a plurality of openings 7 towards the heat dissipation body 3, and the fan is electrically connected to the control module 5, as shown in FIGS. 8 to 10.

According to above-mentioned content, the present invention provides two embodiments, one of the two embodiments includes liquid contained in the heat dissipation space 2, as shown in FIGS. 1 to 4, the thermoelectric cooling chip 41 is electrically connected to the control module 5, so that the control module 5 can transfer electrical power to the thermoelectric cooling chip 41, and the thermoelectric cooling chip 41 can convert electrical power into heating energy or cooling energy and then transfer the heating energy or cooling energy to the heatsink 43 through the thermal conduction body 42, and the sink portion 432 of the heatsink 43 can transfer the heating energy or cooling energy to the liquid of the heat dissipation space 2, thereby achieving the effect of adjusting temperature. Furthermore, when the sink portion 432 of the heatsink 43 is in contact with the liquid in the heat dissipation space 2, the contact area therebetween can be increased and heat transfer efficiency can be improved, so that temperature of the liquid can be adjusted more quickly.

Another embodiment uses the fan as the heat dissipation body 3, as shown in FIGS. 8 to 10, the thermoelectric cooling chip 41 is electrically connected to the control module 5, so that the control module 5 can transfer electrical power to the thermoelectric cooling chip 41, and the thermoelectric cooling chip 41 can convert electrical power into heating energy or cooling energy, and then transfer the heating energy or cooling energy to the heatsink 43 through the thermal conduction body 42, and the sink portion 432 of the heatsink 43 can transfer the heating energy or cooling energy to the heat dissipation space 2, and the fan in the heat dissipation space 2 can be used to transfer heating energy or cooling energy to the outside through the plurality of openings 7.

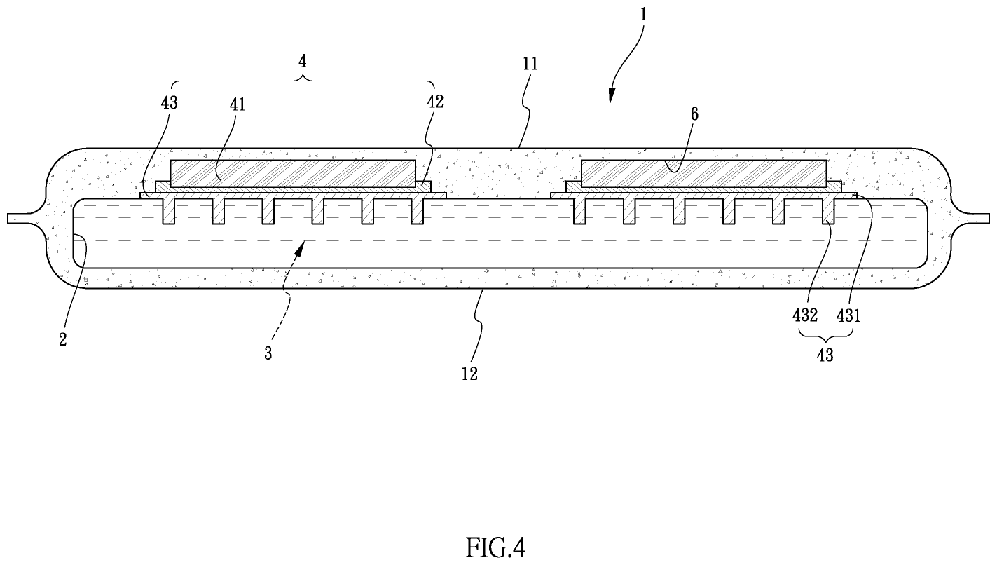

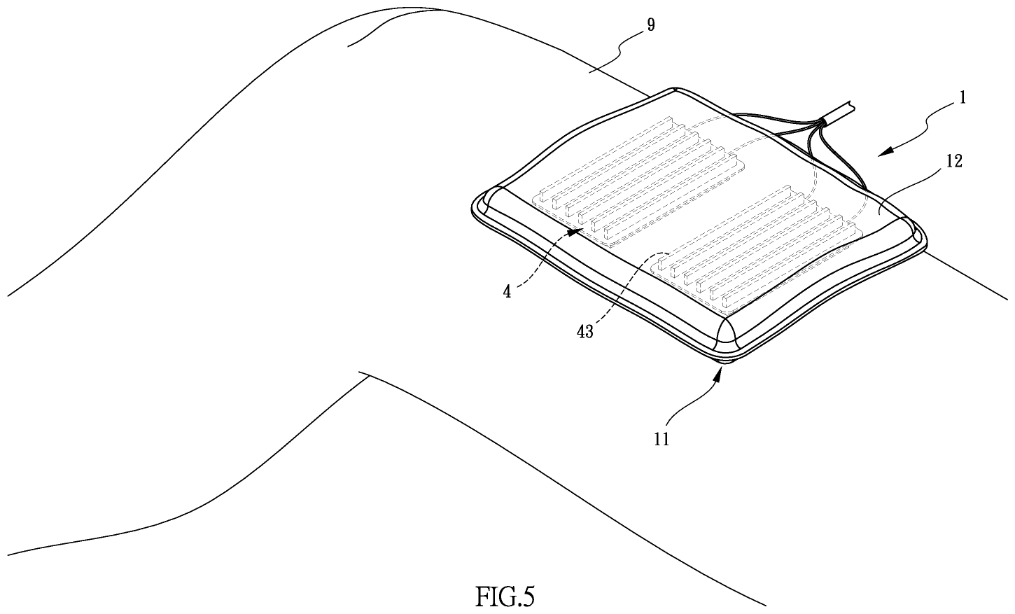

Please refer to FIGS. 2 and 4, the thermal conduction body 42 is disposed between the thermoelectric cooling chip 41 and the heatsink 43, and configured to increase contact area to improve heat transfer efficiency, so as to achieve effect of adjusting temperature more quickly.

According to above-mentioned content, the at least one device body 1 can be flexible, for example, the at least one device body 1 can be silica gel.

In an embodiment, the thermal conduction body 42 can be paste, such as thermal paste.

In an embodiment, the thermal conduction body 42 can be sheet-shaped.

Please refer to FIGS. 11 and 12. The device body 1 can include a vibrating motor 8 disposed thereon and electrically connected to the control module 5. The vibrating motor 8 is able to provide a vibration massage effect.

Please refer to FIGS. 13 and 14. The device body 1 has a side serving as a usage surface 11, and other side serving as a heat dissipation surface 12. The at least one device body 1 can further comprises an electrotherapy device 13; furthermore, the at least one device body 1 can also include the vibrating motor 8. The electrotherapy device 13 is disposed on the usage surface 11 of the device body 1 and electrically connected to the control module 5. The electrotherapy device 13 is able to provide electrotherapy effect.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. A health device, comprising:
at least one device body comprising a heat dissipation space enclosed in the inside thereof, a heat dissipation body disposed in the heat dissipation space, and at least one temperature adjusting device disposed thereon, wherein a part of the at least one temperature adjusting device faces the heat dissipation space, the at least one temperature adjusting device comprises a thermoelectric cooling chip, a thermal conduction body and a heatsink, the thermal conduction body is disposed between the thermoelectric cooling chip and the heatsink, the heatsink comprises a plate portion and a sink portion, a side of the plate portion is abutted with the thermal conduction body, and the sink portion is extended from other side of the plate portion and in contact with the heat dissipation body of the heat dissipation space; and
a control module electrically connected to the thermoelectric cooling chip of the at least one temperature adjusting device.

2. The health device according to claim 1, wherein the at least one device body has at least one accommodating groove recessed on an inside wall thereof, and the at least one temperature adjusting device is disposed in the accommodating groove.

3. The health device according to claim 1, wherein the at least one device body is flexible, and the at least one device body is silica gel.

4. The health device according to claim 1, wherein the thermal conduction body is paste, and the thermal conduction body is thermal paste.

5. The health device according to claim 1, wherein the thermal conduction body is sheet-shaped.

6. The health device according to claim 1, wherein the heat dissipation body is liquid and the heat dissipation body is water.

7. The health device according to claim 1, wherein the heat dissipation body is a fan, the at least one device body comprises a plurality of openings towards the fan, and the fan is electrically connected to the control module.

8. The health device according to claim 1, wherein the device body comprises a vibrating motor disposed thereon and electrically connected to the control module.

9. The health device according to claim 1, wherein the number of the at least one device body is multiple, and the control module is electrically connected to the multiple device bodies.

10. The health device according to claim 1, wherein the at least one device body has a side serving as a usage surface, and other side serving as a heat dissipation surface, and the health device further comprises an electrotherapy device disposed on the usage surface of the device body and electrically connected to the control module.
